(19) United States
(12) Patent Application Publication
(10) Pub. No.: US 2018/0130552 A1
(43) Pub. Date: May 10, 2018

(54) Injection System and Method

(71) Applicant: Liebel-Flarsheim Company LLC, Cincinnati, OH (US)

(72) Inventor: Robert J. Ziemba, Cincinnati, OH (US)

(21) Appl. No.: 15/643,270

(22) Filed: Jul. 6, 2017

Related U.S. Application Data
(63) Continuation of application No. 14/131,764, filed on Jan. 9, 2014, now abandoned.
(60) Provisional application No. 61/507,682, filed on Jul. 14, 2011.

(51) Int. Cl. A61M 5/145

(52) U.S. Cl. CPC G16H 20/17; A61M 5/14546; A61M 2205/50; G16H 80/00; A61M 2205/60; A61M 2205/52

(57) ABSTRACT

An injection data management system (172) is disclosed. Injection-related data (182) is stored on the injection data management system (172) on a customer-by-customer (122) basis. A given customer (122) may communicate with the injection data management system (172) over the Internet (160) using an appropriate data communications device (124). Upon providing appropriate authenticating data (146) to the system (172), a customer (122) can access, view, and/or assess its own injection-related data (182) that is stored on the system (172) over the Internet (160).

### CROSS-REFERENCE TO RELATED APPLICATIONS

This is a continuation of U.S. patent application Ser. No. 14/131,764, entitled “INJECTION DATA MANAGEMENT SYSTEM AND METHOD,” filed on Jan. 9, 2014, which is a 371 National Phase Patent Application of International Patent App. No. PCT/US12/45358, entitled “INJECTION DATA MANAGEMENT SYSTEM AND METHOD,” filed on Jul. 3, 2012, which claims priority to U.S. Provisional Patent App. No. 61/507,682, entitled “INJECTION DATA MANAGEMENT SYSTEM AND METHOD,” filed on Jul. 14, 2011, the entire contents of which are hereby incorporated by reference in their entireties herein.

### FIELD OF THE INVENTION

The present invention generally relates to the field of injection systems and, more particularly, to the field of management of data related to injection systems.

### BACKGROUND

Various medical procedures require that one or more medical fluids be injected into a patient. For example, medical imaging procedures oftentimes involve the injection of contrast media into a patient, possibly along with saline and/or other fluids. Other medical procedures involve injecting one or more fluids into a patient for therapeutic purposes. Power injectors may be used for these types of applications.

A power injector generally includes what is commonly referred to as a powerhead. One or more syringes may be mounted to the powerhead in various manners (e.g., detachably; rear-loading; front-loading; side-loading). Each syringe typically includes what may be characterized as a syringe plunger, piston, or the like. Each such syringe plunger is designed to interface with (e.g., contact and/or temporarily interconnect with) an appropriate syringe plunger driver that is incorporated into the powerhead, such that operation of the syringe plunger driver axially advances the associated syringe plunger inside and relative to a barrel of the syringe. One typical syringe plunger driver is in the form of a ram that is mounted on a threaded lead or drive screw. Rotation of the drive screw in one rotational direction advances the associated ram in one axial direction, while rotation of the drive screw in the opposite rotational direction advances the associated ram in the opposite axial direction.

### SUMMARY

A first aspect of the present invention is embodied by a method of managing injection-related data. A first injection device is operated, and first injection-related data is generated and is associated in at least some manner with this operation of the first injection device. This first injection-related data is output to the Internet in association with an Internet address for an injection data management system. A first request for a first customer injection-related data selection is output to the Internet in association with an Internet address for the injection data management system. The first customer injection-related data selection is received via the Internet and in response to the first request, where this first customer injection-related data selection is based at least in part on the first injection-related data.

A number of feature refinements and additional features are applicable to the first aspect of the present invention. These feature refinements and additional features may be used individually or in any combination. As such, each of the following features that will be discussed may be, but are not required to be, used with any other feature or combination of features of the first aspect. The following discussion is applicable to the first aspect, up to the start of the discussion of a second aspect of the present invention. Generally, “injection-related data” is data that relates in at least some manner to operation of an injection device, and that is output to the Internet (e.g., for storage on the injection data management system). “Customer injection-related data” is the compilation of a customer's injection-related data that is stored on the injection data management system. A “customer injection-related data selection” is that portion of the customer injection-related data that is defined by or corresponds with a particular request formulated by the customer.

The method of the first aspect may be characterized as how a customer interfaces/interacts with an injection data management system. A “customer” may be characterized as an owner/operator of an injection device or may be an owner/operator of a plurality of injection devices. Representative customers for purposes of the present invention include without limitation owners/operators of hospitals, clinics, stand-alone imaging centers, mobile imaging vehicles (e.g., vans, trucks, tractor-trailers), or the like. Any outputs to the Internet in accordance with the first aspect (e.g., an output of injection-related data; a request for a customer injection-related data selection) may include an appropriate Internet address (e.g., a server address for the injection data management system).

It should be appreciated that the first aspect does not itself require the injection data management system, but instead is directed to “customer-side” activities in relation to such an injection data management system. For instance, the injection data management system may be maintained by a third party (e.g., one or more actions by such a third party are not required by the first aspect), may service one or more customers (e.g., different hospitals, clinics, imaging centers, mobile imaging vehicles (e.g., vans, trucks, tractor-trailers), or the like), and may provide a capability for a customer to conveniently store, access, and/or assess the customer's injection-related data. Such an injection data management system may be of any appropriate configuration that may communicate with one or more customers via the Internet. For instance, this injection data management system may be characterized as a platform in the form of a single machine or multiple machines that are distributed in any appropriate manner, where one or more machines of the platform may include one or more web-based applications, software, firmware, or any combination thereof. In one embodiment, the injection data management system may be in the form of one or more servers interconnected to the Internet. Any appropriate web-based architecture may be utilized by the injection data management system.

The injection data management system may be characterized as a centralized repository for injection-related data for use by one or more customers. One or more customers may remotely access the injection data management system by appropriately addressing the same to both transmit (over the Internet) their own injection-related data to the injection data management system for storage, as well as to receive at least some of their own injection-related data (over the Internet) that has been previously stored on the injection data management system. The outputting of the first injection-related data to the Internet and the outputting of the first request for a first customer injection-related data selection to the Internet in the case of the first aspect each may be characterized as being executed outside of the injection data management system, by remotely accessing the injection data management system over the Internet, or both.

The outputting of the first injection-related data to the Internet may be associated with a first Internet address for the injection data management system (e.g., the injection data management system may be accessed via such a first Internet address), and including where multiple customers may access the injection data management system over the Internet using a common Internet address. Regarding the outputting of the first injection-related data: 1) the outputting of the first injection-related data to the Internet may include designating the first injection-related data for uploading to the injection data management system over the Internet (e.g., transmitting data over the Internet from a peripheral device (a device that is not part of the injection data management system) to the injection data management system); 2) the first injection-related data may be actually uploaded to the injection data management system by outputting the first injection-related data to the Internet in association with an Internet address for the injection data management system; and/or 3) the first injection-related data may be transmitted to a server of the injection data management system (e.g., for centralized storage) by outputting the first injection-related data to the Internet in association with an Internet address for the injection data management system.

The outputting of the first request for a customer injection-related data selection to the Internet may be associated with a first Internet address for the injection data management system (e.g., the injection data management system may be accessed via such a first Internet address). The receipt of the first customer injection-related data selection over the Internet may include downloading the first customer injection-related data selection from the injection data management system over the Internet (e.g., transmitting data over the Internet from the injection data management system to a peripheral device (a device that is not part of the injection data management system)). In one embodiment, the first request for a first customer injection-related data selection is executed at some point in time after the first injection-related data has been output to the Internet in association with an Internet address for the injection data management system.

The first injection device may be of any appropriate size, shape, configuration, and/or type, for instance in the form of a power injector (e.g., a single-head configuration; a dual-head configuration). The first injection-related data may be output directly from the first injection device to the Internet (e.g., for storage on the injection data management system) in any appropriate manner and on any appropriate basis. Data relating to operation of the first injection device may be stored on the first injection device, and the first injection-related data may be output directly from the first injection device to the Internet (e.g., for storage on the injection data management system). Data relating to operation of the first injection device may be transferred to and stored on a first data storage device that is operatively interconnected (e.g., continually, or at regular or irregular intervals) with the first injection device, and the first injection-related data may be output to the Internet (e.g., for storage on the injection data management system) directly from this first data storage device. As will be discussed in more detail below, the first aspect may be applicable to managing injection-related data for a customer having one or more injection devices at one or more locations within a given facility, at one or more facilities, or both. In this regard, multiple injection devices could be operatively interconnected with a common data storage device (e.g., the first data storage device), and injection-related data could be output directly from this data storage device to the Internet for purposes of receiving a specified customer injection-related data selection back that relates in at least some respect to the operation of one or more of the customer's injection devices.

The first injection-related data that is generated in relation to the operation of the first injection device may be of any appropriate type. For instance, the first injection-related data may be in the form of a programmed and achieved fluid injection rate, a date of an injection, a programmed and achieved volume of fluid (e.g. contrast media) injected, a type (e.g., brand) of fluid injected, contrast media usage, minimum/average/maximum fluid pressure generated during an injection, fluid injection rate as a function of time during an injection, injection pressure as a function of time during an injection, a lot number of the fluid injected, an iodine-content of the fluid injected, an identity of the facility responsible for manufacturing and/or packaging the fluid injected, a manufacturing date of the fluid injected, an expiration date of the fluid injected, the National Drug Code (NDC) of the fluid injected, a volume of fluid remaining and subsequently discarded after an injection procedure (e.g., contrast media remaining in a syringe at the completion of an injection procedure), or any combination thereof. With regard to the minimum, average, and maximum pressures noted above, the same may relate to the pressure that was generated during a single injection procedure (e.g., executed in accordance with a first injection protocol or a programmed protocol) using the first injection device.

At least some of the first injection-related data may be retrieved directly from the first injection device, at least some of the first injection-related data may be retrieved other than from the first injection device, or any combination thereof. For instance and in the latter regard, a data reader of any appropriate type (e.g., an RFID reader) may be used to read one or more data tags (or other data storage devices, for instance an RFID tag) associated with one or more fluid containers (e.g., syringes) that may be used by the first injection device. In one embodiment, such a data reader is used to identify one or more aspects of the fluid (e.g., type, concentration, preparation date, initial volume of fluid in the fluid container) to be delivered by the first injection device. The injection data management system may also be configured to allow customers to store other injection-related data, for instance quantities of contrast media that have been ordered and/or received by a customer (e.g., for inventory management purposes).

A request for a customer injection-related data selection may be characterized as allowing a customer to access, review, analyze, and/or compile all or any portion of its own injection-related data. All results of any such request may be characterized as a “customer injection-related data selection.” Representative customer injection-related data selections include without limitation: 1) the number of injection procedures run on one or more of the customer's injection devices, at one or more of the customer's facilities, or both; 2) the number of a particular type of injection procedure (e.g., a particular injection protocol) run on one or more of the customer's injection devices, at one or more of the customer's facilities, or both; 3) the flow rate (minimum, maximum, and/or average) used for one or more (e.g., a particular type of) injection procedures run on one or more of the customer's injection devices, at one or more of the customer's facilities, or both; 4) the average fluid injection rate employed by one or more of the customer's injection devices, or optionally specific for various injection procedures; 5) the actual and/or average volumes of fluid (e.g. contrast media) utilized by one or more of the customer's injection devices for a given time period (e.g., daily, weekly, monthly, quarterly, yearly); 6) the volume of fluid remaining and subsequently discarded after an injection procedure (e.g., contrast media remaining in a syringe at the completion of an injection procedure); 7) the pressure (minimum, maximum, and/or average) generated during the execution of a particular type of injection procedure (e.g., in accordance with a first injection protocol or otherwise) run on a particular one of the customer's injection devices; and 8) the pressure (minimum, maximum, and/or average) generated during the execution of multiple injection procedures (e.g., each executed in accordance with a common injection protocol or otherwise) run on one or more of the customer's injection devices, at one or more of the customer's facilities, or both.

A request for a customer injection-related data selection, that is output to the Internet in association with an Internet address for the injection data management system, may be of any appropriate type and/or form. The request may be in the form of one or more filter selections, parameters, or the like for purposes of defining the desired customer injection-related data selection. An electronic form (e.g., via one or more web-based applications and/or a web-based interface) may be used by a customer (e.g., on an appropriate communications device) to formulate a request for the desired customer injection-related data selection. A customer could request injection-related data regarding one or more injection devices at one or more of its facilities. A customer could also request injection-related data regarding one or more of its facilities (e.g., contrast media usage at one or more of its facilities; contrast media inventory at one or more of its facilities).

A customer injection-related data selection that is received in response to a request may be of any appropriate type and in any appropriate form (e.g., an electronic report viewable on an appropriate communications device utilizing one or more web-based applications and/or a web-based interface). Any such customer injection-related data selection may be of any appropriate content as well. For instance, a customer injection-related data selection may be customer contrast media usage data (e.g., how much contrast media has been used by one or more injection devices), may be customer contrast media inventory data (e.g., how much contrast media remains in inventory at one or more facilities owned and/or operated by a given customer), or both. Any such customer injection-related data selection could be specific to a single injection device (e.g., the first injection device) or could be based upon one or more injection devices at the same location/facility, one or more injection devices at different locations/facilities, or both. Consider the case where a second injection device is operated, and where second injection-related data is generated that is at least somehow associated with this operation of the second injection device for purposes of the first aspect. This second injection-related data may be output to the Internet in association with an Internet address for the injection data management system. A given customer injection-related data selection may not only be based upon the first injection-related data, but the second injection-related data as well. Another option is for the first aspect to include the outputting of a second request for a second customer injection-related data selection to the Internet in association with an Internet address for the injection data management system. The second customer injection-related data selection may be received via the Internet and in response to this second request, where this second customer injection-related data selection is based at least in part on the second injection-related data (e.g., the second request could be specific to the second injection device).

One or more communications devices, of any appropriate type and/or at any appropriate location, may be used by a given customer to output a request for a customer injection-related data selection, to receive the corresponding results (i.e., a customer injection-related data selection), or both. For instance, a customer could use a laptop computer, a netbook, a desktop computer, a tablet PC, a personal digital assistant or PDA, a Smart Phone, or the like. One or more web-based interfaces may be utilized to output requests, to view the corresponding results, or both, including without limitation using any appropriate Internet-access device. A customer injection-related data selection may be printed out and/or saved locally using an appropriate communications device.

Various customer personnel could utilize the method of this first aspect from any appropriate Internet-access device and from any appropriate location. For instance, a radiologist could output a request for a customer injection-related data selection from (and review the corresponding results on) a PDA. A clinician could output a request for a customer injection-related data selection from (and review the corresponding results on) a desktop/laptop computer located in an imaging suite (or any other appropriate location). A department manager could output a request for a customer injection-related data selection from (and review the corresponding results on) a desktop/laptop computer located in their office (or any other appropriate location). An administrator could output a request for a customer injection-related data selection from (and review the corresponding results on) a desktop/laptop computer from home (or any other appropriate location).

Customer identification information (of any appropriate type and in any appropriate form) may be associated with and/or linked to injection-related data that is output to the Internet in association with an Internet address for the injection data management system and that at least somehow relates to operation of one or more customer injection devices. Access to customer injection-related data on the injection data management system may be provided on a customer-by-customer basis. For instance, although a given customer may be provided access to all or at least a portion of its injection-related data stored on the injection data management system, preferably a given customer is not able to access any injection-related data of another customer stored on the injection data management system. In this regard and in one embodiment, the first aspect may include entering customer authenticating data (e.g., providing a username and/or password), outputting this customer authenticating data to the Internet in association with an Internet address for the injection data management system, or both. Receipt of a customer injection-related data selection in response to a request may be limited to the situation where the authenticating data from the customer matches authentication data that is associated with the customer injection-related data stored on the injection data management system.

A second aspect of the present invention is embodied by a method of managing injection-related data. First injection-related data is received and stored on an injection data management system. This first injection-related data pertains to a first injection device that is associated with a first customer. Second injection-related data is received and stored on the injection data management system. This second injection-related data pertains to a second injection device that is associated with a second customer. Access to customer injection-related data stored by or on the injection data management system is provided on a customer-by-customer basis.

A number of feature refinements and additional features are applicable to the second aspect of the present invention. These feature refinements and additional features may be used individually or in any combination. As such, each of the following features that will be discussed may be, but are not required to be, used with any other feature or combination of features of the second aspect. The following discussion is applicable to the second aspect, up to the start of the discussion of a third aspect of the present invention. Initially, each of the first and second injection devices, as well as the first and second injection-related data, may be in accordance with the corresponding discussion presented above on the first aspect of the present invention.

The method of the second aspect may be characterized as how an injection data management system interfaces/interacts with a plurality of different customers that each may utilize the injection data management system. However, the second aspect does not itself require the plurality of customers, but instead is directed to what may be characterized as owner/operator activities of such a system. For instance, the injection data management system may be maintained by a third party, may service one or more customers (e.g., different hospitals, clinics, stand-alone imaging centers, mobile imaging vehicles (e.g., vans, trucks, tractor-trailers), or the like), and may provide a capability for a customer to conveniently store, access, and/or assess their own injection-related data. Such an injection data management system may be of any appropriate configuration that may communicate with one or more customers, for instance via the Internet. For instance, this injection data management system may be characterized as a platform in the form of a single machine or may be distributed across multiple machines, where one or more machines of the platform may include one or more web-based applications, software, firmware, or any combination thereof. In one embodiment, the injection data management system may be in the form of one or more servers interconnected to the Internet. Any appropriate web-based architecture may be utilized by the injection data management system.

Communication with the injection data management system may be via the Internet (e.g., the injection data management system may be remotely accessible by one or more Internet addresses). The injection data management system may utilize at least one server on the Internet. One or more server addresses may be utilized by the injection data management system. One or more web-based interfaces may be utilized by the injection data management system to allow customers to communicate with the injection data management system via an appropriate Internet-access device.

Access to injection-related data stored by the injection data management system may be provided on a customer-by-customer basis in the case of the second aspect. The first injection-related data may be stored in association with the first customer, while the second injection-related data may be stored in association with the second customer (e.g., injection-related data stored on the injection data management system may be linked in any appropriate to the relevant customer). The injection data management system may be configured such that the first customer only has access to injection-related data that is associated with the first customer by the injection data management system (e.g., over the Internet, for instance in accordance with the first aspect). Similarly, the injection data management system may be configured such that the second customer only has access to injection-related data that is associated with the second customer by the injection data management system (e.g., over the Internet, for instance in accordance with the first aspect).

Customer identification information may be associated with injection-related data that is stored by the injection data management system. Access to customer injection-related data on the injection data management system may be provided on a customer-by-customer basis. For instance, although a given customer may be provided access to all or at least a portion of its injection-related data stored on the injection data management system, preferably a given customer is not able to access any injection-related data of another customer that is stored on the injection data management system. In this regard and in one embodiment, the second aspect may include storing customer authentication data (e.g., providing a username and/or password) in conjunction with customer injection-related data. Outputting information in response to a request for a customer injection-related data selection may be limited to the situation where the authentication data stored on the injection data management system matches authenticating data that is provided along with and/or otherwise accompanies the noted request.

The injection data management system may store injection-related data on one or more injection devices at one or more customer facilities, all on a customer-by-customer basis. The injection data management system may receive a request for a customer injection-related data selection from one of its customers. Such a request may be in accordance with the discussion presented above on the first aspect. A customer injection-related data selection (e.g., in accordance with the discussion of the same for the case of the first aspect) may be compiled and then output (e.g., to the Internet) by the injection data management system.

A third aspect of the present invention is embodiment by an injection data management system. The system may include an appropriate platform. First customer injection-related data is stored on the platform in association with a first customer, and second customer injection-related data is stored on the platform in association with the second customer. First authentication data is also stored on the platform and is appropriately linked to the first customer. Similarly, second authentication data is stored on the platform and is appropriately linked to the second customer. The system further includes a web-based customer interface.

A number of feature refinements and additional features are applicable to the third aspect of the present invention. These feature refinements and additional features may be used individually or in any combination. As such, each of the following features that will be discussed may be, but are not required to be, used with any other feature or combination of features of the third aspect. The following discussion is applicable to the third aspect. Initially, the first and second injection-related data may be in accordance with the corresponding discussion presented above on the first aspect of the present invention.

The platform of the third aspect may be in the form of a single machine or may be distributed across multiple machines, where one or more machines of the platform may include one or more web-based applications, software, firmware, or any combination thereof. In one embodiment, the injection data management system may be in the form of one or more servers interconnected to the Internet. Any appropriate web-based architecture may be utilized by the injection data management system.

The injection-related data that is stored on the injection data management system may be as discussed above in relation to each of the first and second aspects. Moreover, the customer authentication data that is stored on the injection data management system may be of any appropriate type and/or form, for instance in the form of a customer ID or username, customer password, a combination thereof, or the like.

Any feature of any other various aspects of the present invention that is intended to be limited to a “singular” context or the like will be clearly set forth herein by terms such as “only,” “single,” “limited to,” or the like. Merely introducing a feature in accordance with commonly accepted antecedent basis practice does not limit the corresponding feature to the singular (e.g., indicating that a power injector includes “a syringe” alone does not mean that the power injector includes only a single syringe). Moreover, any failure to use phrases such as “at least one” also does not limit the corresponding feature to the singular (e.g., indicating that a power injector includes “a syringe” alone does not mean that the power injector includes only a single syringe). Use of the phrase “at least generally” or the like in relation to a particular feature encompasses the corresponding characteristic and insubstantial variations thereof (e.g., indicating that a syringe barrel is at least generally cylindrical encompasses the syringe barrel being cylindrical). Finally, a reference of a feature in conjunction with the phrase “in one embodiment” does not limit the use of the feature to a single embodiment.

Any “logic” that may be utilized by any of the various aspects of the present invention may be implemented in any appropriate manner, including without limitation in any appropriate software, firmware, or hardware, using one or more platforms, using one or more processors, using memory of any appropriate type, using any single computer of any appropriate type or a multiple computers of any appropriate type and interconnected in any appropriate manner, or any combination thereof. This logic may be implemented at any single location or at multiple locations that are interconnected in any appropriate manner (e.g., via any type of network).

Any power injector that may be utilized to provide a fluid discharge may be of any appropriate size, shape, configuration, and/or type. Any such power injector may utilize one or more syringe plunger drivers of any appropriate size, shape, configuration, and/or type, where each such syringe plunger driver is capable of at least bi-directional movement (e.g., a movement in a first direction for discharging fluid; a movement in a second direction for accommodating a loading and/or drawing of fluid and/or so as to return to a position for a subsequent fluid discharge operation), and where each such syringe plunger driver may interact with its corresponding syringe plunger in any appropriate manner (e.g., by mechanical contact; by an appropriate coupling (mechanical or otherwise)) so as to be able to advance the syringe plunger in at least one direction (e.g., to discharge fluid). Each syringe plunger driver may utilize one or more drive sources of any appropriate size, shape, configuration, and/or type. Multiple drive source outputs may be combined in any appropriate manner to advance a single syringe plunger at a given time. One or more drive sources may be dedicated to a single syringe plunger driver, one or more drive sources may be associated with multiple syringe plunger drivers (e.g., incorporating a transmission of sorts to change the output from one syringe plunger to another syringe plunger), or a combination thereof. Representative drive source forms include a brushed or brushless electric motor, a hydraulic motor, a pneumatic motor, a piezoelectric motor, or a stepper motor.

Any such power injector may be used for any appropriate application where the delivery of one or more medical fluids is desired, including without limitation any appropriate medical imaging application (e.g., computed tomography or CT imaging; magnetic resonance imaging or MRI; single photon emission computed tomography or SPECT imaging; positron emission tomography or PET imaging; X-ray imaging; angiographic imaging; optical imaging; ultrasound imaging) and/or any appropriate medical diagnostic and/or therapeutic application (e.g., injection of chemotherapy, pain management, etc.). Any such power injector may be used in conjunction with any component or combination of components, such as an appropriate imaging system (e.g., a CT scanner). For instance, information could be conveyed between any such power injector and one or more other components (e.g., scan delay information, injection start signal, injection rate).

Any appropriate number of syringes may be utilized with any such power injector in any appropriate manner (e.g., detachably; front-loaded; rear-loaded; side-loaded), any appropriate medical fluid may be discharged from a given syringe of any such power injector (e.g., contrast media, therapeutic fluid, a radiopharmaceutical, saline, and any combination thereof), and any appropriate fluid may be discharged from a multiple syringe power injector configuration in any appropriate manner (e.g., sequentially, simultaneously), or any combination thereof. In one embodiment, fluid discharged from a syringe by operation of the power injector is directed into a conduit (e.g., medical tubing set), where this conduit is fluidly interconnected with the syringe in any appropriate manner and directs fluid to a desired location (e.g., to a catheter that is inserted into a patient for injection). Multiple syringes may discharge into a common conduit (e.g., for provision to a single injection site), or one syringe may discharge into one conduit (e.g., for provision to one injection site), while another syringe may discharge into a different conduit (e.g., for provision to a different injection site). In one embodiment, each syringe includes a syringe barrel and a plunger that is disposed within and movable relative to the syringe barrel. This plunger may interface with the power injector's syringe plunger drive assembly such that the syringe plunger drive assembly is able to advance the plunger in at least one direction, and possibly in two different, opposite directions.

### BRIEF DESCRIPTION OF THE FIGURES

FIG. 1 is a schematic of one embodiment of a power injector.

FIG. 2A is a perspective view of one embodiment of a portable stand-mounted, dual-head power injector.

FIG. 2B is an enlarged, partially exploded, perspective view of a powerhead used by the power injector of FIG. 2A.

FIG. 2C is a schematic of one embodiment of a syringe plunger drive assembly used by the power injector of FIG. 2A.

FIG. 3 is one embodiment of an injection data management architecture.

FIG. 4 is a representative implementation of the injection data management architecture of FIG. 3.

FIG. 5 is a schematic of one configuration for the injection data management system that may be used by the injection data management architectures of FIGS. 3 and 4.

FIG. 6 is a schematic based upon the injection data management architecture of FIG. 3, and for the case of a customer having multiple facilities.

FIG. 7 is a flowchart of one embodiment of an injection-related data storage protocol that may be used by the injection data management architectures of FIGS. 3 and 4.

FIG. 8 is a flowchart of one embodiment of an injection-related data request protocol that may be used by the injection data management architectures of FIGS. 3 and 4.

### DETAILED DESCRIPTION

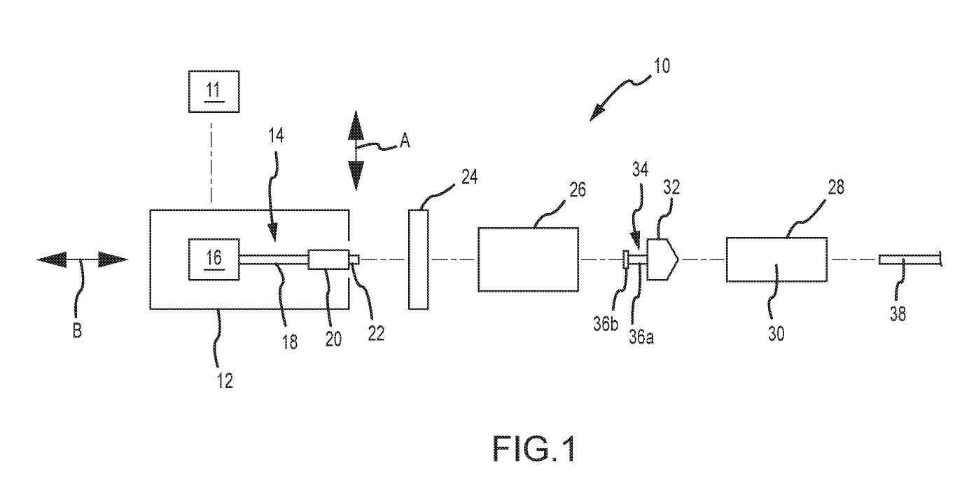

FIG. 1 presents a schematic of one embodiment of a power injector 10 having a powerhead 12. One or more graphical user interfaces or GUIs 11 may be associated with the powerhead 12. Each GUI 11: 1) may be of any appropriate size, shape, configuration, and/or type; 2) may be operatively interconnected with the powerhead 12 in any appropriate manner; 3) may be disposed at any appropriate location; 4) may be configured to provide any of the following functions: controlling one or more aspects of the operation of the power injector 10; inputting/editing one or more parameters associated with the operation of the power injector 10; and displaying appropriate information (e.g., associated with the operation of the power injector 10); or 5) any combination of the foregoing. Any appropriate number of GUIs 11 may be utilized. In one embodiment, the power injector 10 includes a GUI 11 that is incorporated by a console that is separate from but which communicates with the powerhead 12. In another embodiment, the power injector 10 includes a GUI 11 that is part of the powerhead 12. In yet another embodiment, the power injector 10 utilizes one GUI 11 on a separate console that communicates with the powerhead 12, and also utilizes another GUI 11 that is on the powerhead 12. Each GUI 11 could provide the same functionality or set of functionalities, or the GUIs 11 may differ in at least some respect in relation to their respective functionalities.

A syringe 28 may be installed on the powerhead 12 and, when installed, may be considered to be part of the power injector 10. Some injection procedures may result in a relatively high pressure being generated within the syringe 28. In this regard, it may be desirable to dispose the syringe 28 within a pressure jacket 26. The pressure jacket 26 is typically associated with the powerhead 12 in a manner that allows the syringe 28 to be disposed therein as a part of or after installing the syringe 28 on the powerhead 12. The same pressure jacket 26 will typically remain associated with the powerhead 12, as various syringes 28 are positioned within and removed from the pressure jacket 26 for multiple injection procedures. The power injector 10 may eliminate the pressure jacket 26 if the power injector 10 is configured/utilized for low-pressure injections and/or if the syringe(s) 28 to be utilized with the power injector 10 is (are) of sufficient durability to withstand high-pressure injections without the additional support provided by a pressure jacket 26. In any case, fluid discharged from the syringe 28 may be directed into a conduit 38 of any appropriate size, shape, configuration, and/or type, which may be fluidly interconnected with the syringe 28 in any appropriate manner, and which may direct fluid to any appropriate location (e.g., to a patient).

The powerhead 12 includes a syringe plunger drive assembly or syringe plunger driver 14 that interacts (e.g., interfaces) with the syringe 28 (e.g., a plunger 32 thereof) to discharge fluid from the syringe 28. This syringe plunger drive assembly 14 includes a drive source 16 (e.g., a motor of any appropriate size, shape, configuration, and/or type, optional gearing, and the like) that powers a drive output 18 (e.g., a rotatable drive screw). A ram 20 may be advanced along an appropriate path (e.g., axial) by the drive output 18. The ram 20 may include a coupler 22 for interacting or interfacing with a corresponding portion of the syringe 28 in a manner that will be discussed below.

The syringe 28 includes a plunger or piston 32 that is movably disposed within a syringe barrel 30 (e.g., for axial reciprocation along an axis coinciding with the double-headed arrow B). The plunger 32 may include a coupler 34. This syringe plunger coupler 34 may interact or interface with the ram coupler 22 to allow the syringe plunger drive assembly 14 to retract the syringe plunger 32 within the syringe barrel 30. The syringe plunger coupler 34 may be in the form of a shaft 36a that extends from a body of the syringe plunger 32, together with a head or button 36b. However, the syringe plunger coupler 34 may be of any appropriate size, shape, configuration, and/or type.

Generally, the syringe plunger drive assembly 14 of the power injector 10 may interact with the syringe plunger 32 of the syringe 28 in any appropriate manner (e.g., by mechanical contact; by an appropriate coupling (mechanical or otherwise)) so as to be able to move or advance the syringe plunger 32 (relative to the syringe barrel 30) in at least one direction (e.g., to discharge fluid from the corresponding syringe 28). That is, although the syringe plunger drive assembly 14 may be capable of bi-directional motion (e.g., via operation of the same drive source 16), the power injector 10 may be configured such that the operation of the syringe plunger drive assembly 14 actually only moves each syringe plunger 32 being used by the power injector 10 in only one direction. However, the syringe plunger drive assembly 14 may be configured to interact with each syringe plunger 32 being used by the power injector 10 so as to be able to move each such syringe plunger 32 in each of two different directions (e.g. in different directions along a common axial path).

Retraction of the syringe plunger 32 may be utilized to accommodate a loading of fluid into the syringe barrel 30 for a subsequent injection or discharge, may be utilized to actually draw fluid into the syringe barrel 30 for a subsequent injection or discharge, or for any other appropriate purpose. Certain configurations may not require that the syringe plunger drive assembly 14 be able to retract the syringe plunger 32, in which case the ram coupler 22 and syringe plunger coupler 34 may not be desired. In this case, the syringe plunger drive assembly 14 may be retracted for purposes of executing another fluid delivery operation (e.g., after another pre-filled syringe 28 has been installed). Even when a ram coupler 22 and syringe plunger coupler 34 are utilized, these components may or may not be coupled when the ram 20 advances the syringe plunger 32 to discharge fluid from the syringe 28 (e.g., the ram 20 may simply “push on” the syringe plunger coupler 34 or directly on a proximal end of the syringe plunger 32). Any single motion or combination of motions in any appropriate dimension or combination of dimensions may be utilized to dispose the ram coupler 22 and syringe plunger coupler 34 in a coupled state or condition, to dispose the ram coupler 22 and syringe plunger coupler 34 in an un-coupled state or condition, or both.

The syringe 28 may be installed on the powerhead 12 in any appropriate manner. For instance, the syringe 28 could be configured to be installed directly on the powerhead 12. In the illustrated embodiment, a housing 24 is appropriately mounted on the powerhead 12 to provide an interface between the syringe 28 and the powerhead 12. This housing 24 may be in the form of an adapter to which one or more configurations of syringes 28 may be installed, and where at least one configuration for a syringe 28 could be installed directly on the powerhead 12 without using any such adapter. The housing 24 may also be in the form of a faceplate to which one or more configurations of syringes 28 may be installed. In this case, it may be such that a faceplate is required to install a syringe 28 on the powerhead 12—the syringe 28 could not be installed on the powerhead 12 without the faceplate. When a pressure jacket 26 is being used, it may be installed on the powerhead 12 in the various manners discussed herein in relation to the syringe 28, and the syringe 28 will then thereafter be installed in the pressure jacket 26.

The housing 24 may be mounted on and remain in a fixed position relative to the powerhead 12 when installing a syringe 28. Another option is to movably interconnect the housing 24 and the powerhead 12 to accommodate installing a syringe 28. For instance, the housing 24 may move within a plane that contains the double-headed arrow A to provide one or more of coupled state or condition and an un-coupled state or condition between the ram coupler 22 and the syringe plunger coupler 34.

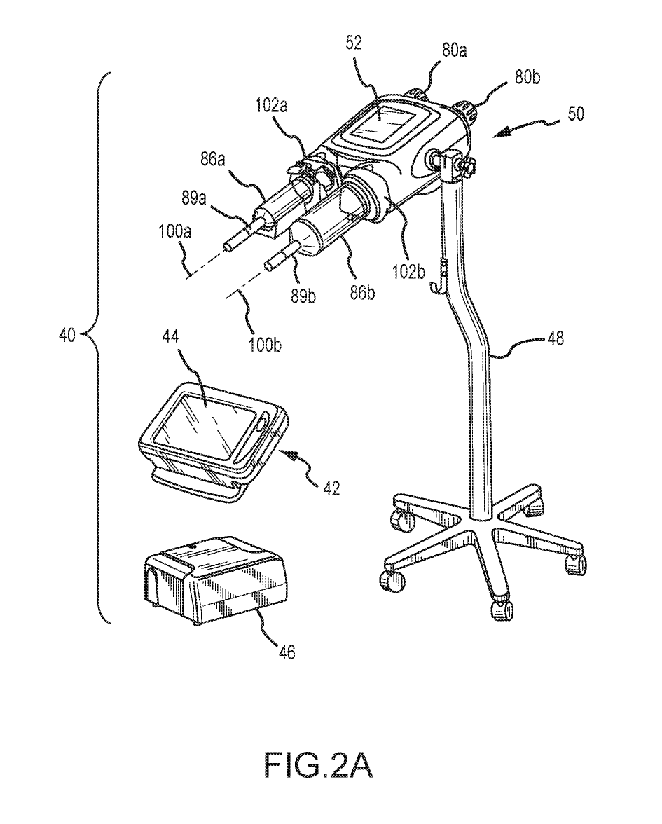

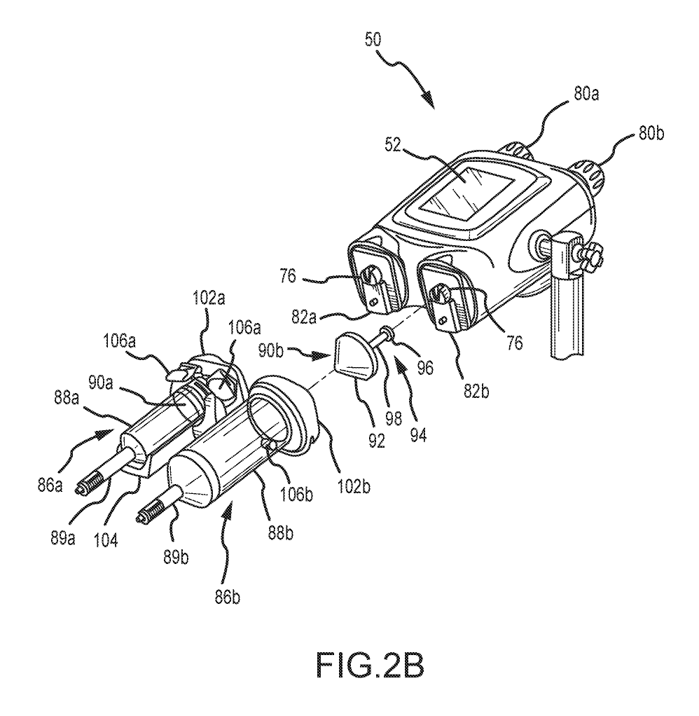

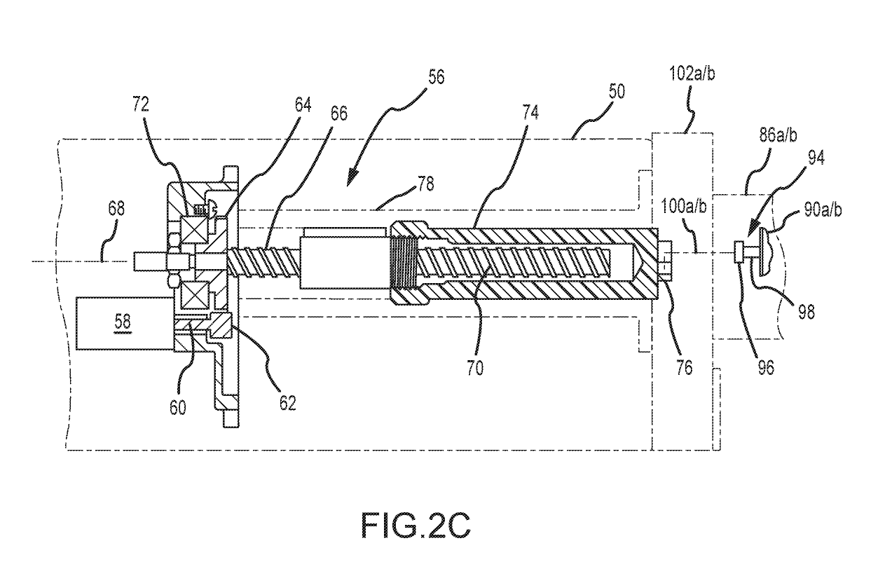

One particular power injector configuration is illustrated in FIG. 2A, is identified by a reference numeral 40, and is at least generally in accordance with the power injector 10 of FIG. 1. The power injector 40 includes a powerhead 50 that is mounted on a portable stand 48. Two syringes 86a, 86b for the power injector 40 are mounted on the powerhead 50. Fluid may be discharged from the syringes 86a, 86b during operation of the power injector 40.

The portable stand 48 may be of any appropriate size, shape, configuration, and/or type. Wheels, rollers, casters, or the like may be utilized to make the stand 48 portable. The powerhead 50 could be maintained in a fixed position relative to the portable stand 48. However, it may be desirable to allow the position of the powerhead 50 to be adjustable relative to the portable stand 48 in at least some manner. For instance, it may be desirable to have the powerhead 50 in one position relative to the portable stand 48 when loading fluid into one or more of the syringes 86a, 86b, and to have the powerhead 50 in a different position relative to the portable stand 48 for performance of an injection procedure. In this regard, the powerhead 50 may be movably interconnected with the portable stand 48 in any appropriate manner (e.g., such that the powerhead 50 may be pivoted through at least a certain range of motion, and thereafter maintained in the desired position).

It should be appreciated that the powerhead 50 could be supported in any appropriate manner for providing fluid. For instance, instead of being mounted on a portable structure, the powerhead 50 could be interconnected with a support assembly, that in turn is mounted to an appropriate structure (e.g., ceiling, wall, floor). Any support assembly for the powerhead 50 may be positionally adjustable in at least some respect (e.g., by having one or more support sections that may be repositioned relative to one or more other support sections), or may be maintained in a fixed position. Moreover, the powerhead 50 may be integrated with any such support assembly so as to either be maintained in a fixed position or so as to be adjustable relative the support assembly.

The powerhead 50 includes a graphical user interface or GUI 52. This GUI 52 may be configured to provide one or any combination of the following functions: controlling one or more aspects of the operation of the power injector 40; inputting/editing one or more parameters associated with the operation of the power injector 40; and displaying appropriate information (e.g., associated with the operation of the power injector 40). The power injector 40 may also include a console 42 and powerpack 46 that each may be in communication with the powerhead 50 in any appropriate manner (e.g., via one or more cables), that may be placed on a table or mounted on an electronics rack in an examination room or at any other appropriate location, or both. The powerpack 46 may include one or more of the following and in any appropriate combination: a power supply for the injector 40; interface circuitry for providing communication between the console 42 and powerhead 50; circuitry for permitting connection of the power injector 40 to remote units such as remote consoles, remote hand or foot control switches, or other original equipment manufacturer (OEM) remote control connections (e.g., to allow for the operation of power injector 40 to be synchronized with the x-ray exposure of an imaging system); and any other appropriate componentry. The console 42 may include a touch screen display 44, which in turn may provide one or more of the following functions and in any appropriate combination: allowing an operator to remotely control one or more aspects of the operation of the power injector 40; allowing an operator to enter/edit one or more parameters associated with the operation of the power injector 40; allowing an operator to specify and store programs for automated operation of the power injector 40 (which can later be automatically executed by the power injector 40 upon initiation by the operator); and displaying any appropriate information relation to the power injector 40 and including any aspect of its operation.

Various details regarding the integration of the syringes 86a, 86b with the powerhead 50 are presented in FIG. 2B. Each of the syringes 86a, 86b includes the same general components. The syringe 86a includes plunger or piston 90a that is movably disposed within a syringe barrel 88a. Movement of the plunger 90a along an axis 100a (FIG. 2A) via operation of the powerhead 50 will discharge fluid from within a syringe barrel 88a through a nozzle 89a of the syringe 86a. An appropriate conduit (not shown) will typically be fluidly interconnected with the nozzle 89a in any appropriate manner to direct fluid to a desired location (e.g., a patient). Similarly, the syringe 86b includes plunger or piston 90b that is movably disposed within a syringe barrel 88b. Movement of the plunger 90b along an axis 100b (FIG. 2A) via operation of the powerhead 50 will discharge fluid from within the syringe barrel 88b through a nozzle 89b of the syringe 86b. An appropriate conduit (not shown) will typically be fluidly interconnected with the nozzle 89b in any appropriate manner to direct fluid to a desired location (e.g., a patient).

The syringe 86a is interconnected with the powerhead 50 via an intermediate faceplate 102a. This faceplate 102a includes a cradle 104 that supports at least part of the syringe barrel 88a, and which may provide/accommodate any additional functionality or combination of functionalities. A mounting 82a is disposed on and is fixed relative to the powerhead 50 for interfacing with the faceplate 102a. A ram coupler 76 of a ram 74 (FIG. 2C), which are each part of a syringe plunger drive assembly or syringe plunger driver 56 (FIG. 2C) for the syringe 86a, is positioned in proximity to the faceplate 102a when mounted on the powerhead 50. Details regarding the syringe plunger drive assembly 56 will be discussed in more detail below in relation to FIG. 2C. Generally, the ram coupler 76 may be coupled with the syringe plunger 90a of the syringe 86a, and the ram coupler 76 and ram 74 (FIG. 2C) may then be moved relative to the powerhead 50 to move the syringe plunger 90a along the axis 100a (FIG. 2A). It may be such that the ram coupler 76 is engaged with, but not actually coupled to, the syringe plunger 90a when moving the syringe plunger 90a to discharge fluid through the nozzle 89a of the syringe 86a.

The faceplate 102a may be moved at least generally within a plane that is orthogonal to the axes 100a, 100b (associated with movement of the syringe plungers 90a, 90b, respectively, and illustrated in FIG. 2A), both to mount the faceplate 102a on and remove the faceplate 102a from its mounting 82a on the powerhead 50. The faceplate 102a may be used to couple the syringe plunger 90a with its corresponding ram coupler 76 on the powerhead 50. In this regard, the faceplate 102a includes a pair of handles 106a. Generally and with the syringe 86a being initially positioned within the faceplate 102a, the handles 106a may be moved to in turn move/translate the syringe 86a at least generally within a plane that is orthogonal to the axes 100a, 100b (associated with movement of the syringe plungers 90a, 90b, respectively, and illustrated in FIG. 2A). Moving the handles 106a to one position moves/translates the syringe 86a (relative to the faceplate 102a) in an at least generally downward direction to couple its syringe plunger 90a with its corresponding ram coupler 76. Moving the handles 106a to another position moves/translates the syringe 86a (relative to the faceplate 102a) in an at least generally upward direction to uncouple its syringe plunger 90a from its corresponding ram coupler 76.

The syringe 86b is interconnected with the powerhead 50 via an intermediate faceplate 102b. A mounting 82b is disposed on and is fixed relative to the powerhead 50 for interfacing with the faceplate 102b. A ram coupler 76 of a ram 74 (FIG. 2C), which are each part of a syringe plunger drive assembly 56 for the syringe 86b, is positioned in proximity to the faceplate 102b when mounted to the powerhead 50. Details regarding the syringe plunger drive assembly 56 again will be discussed in more detail below in relation to FIG. 2C. Generally, the ram coupler 76 may be coupled with the syringe plunger 90b of the syringe 86b, and the ram coupler 76 and ram 74 (FIG. 2C) may be moved relative to the powerhead 50 to move the syringe plunger 90b along the axis 100b (FIG. 2A). It may be such that the ram coupler 76 is engaged with, but not actually coupled to, the syringe plunger 90b when moving the syringe plunger 90b to discharge fluid through the nozzle 89b of the syringe 86b.

The faceplate 102b may be moved at least generally within a plane that is orthogonal to the axes 100a, 100b (associated with movement of the syringe plungers 90a, 90b, respectively, and illustrated in FIG. 2A), both to mount the faceplate 102b on and remove the faceplate 102b from its mounting 82b on the powerhead 50. The faceplate 102b also may be used to couple the syringe plunger 90b with its corresponding ram coupler 76 on the powerhead 50. In this regard, the faceplate 102b may include a handle 106b. Generally and with the syringe 86b being initially positioned within the faceplate 102b, the syringe 86b may be rotated along its long axis 100b (FIG. 2A) and relative to the faceplate 102b. This rotation may be realized by moving the handle 106b, by grasping and turning the syringe 86b, or both. In any case, this rotation moves/translates both the syringe 86b and the faceplate 102b at least generally within a plane that is orthogonal to the axes 100a, 100b (associated with movement of the syringe plungers 90a, 90b, respectively, and illustrated in FIG. 2A). Rotating the syringe 86b in one direction moves/translates the syringe 86b and faceplate 102b in an at least generally downward direction to couple the syringe plunger 90b with its corresponding ram coupler 76. Rotating the syringe 86b in the opposite direction moves/translates the syringe 86b and faceplate 102b in an at least generally upward direction to uncouple its syringe plunger 90b from its corresponding ram coupler 76.

As illustrated in FIG. 2B, the syringe plunger 90b includes a plunger body 92 and a syringe plunger coupler 94. This syringe plunger coupler 94 includes a shaft 98 that extends from the plunger body 92, along with a head 96 that is spaced from the plunger body 92. Each of the ram couplers 76 includes a larger slot that is positioned behind a smaller slot on the face of the ram coupler 76. The head 96 of the syringe plunger coupler 94 may be positioned within the larger slot of the ram coupler 76, and the shaft 98 of the syringe plunger coupler 94 may extend through the smaller slot on the face of the ram coupler 76 when the syringe plunger 90b and its corresponding ram coupler 76 are in a coupled state or condition. The syringe plunger 90a may include a similar syringe plunger coupler 94 for interfacing with its corresponding ram coupler 76.

The powerhead 50 is utilized to discharge fluid from the syringes 86a, 86b in the case of the power injector 40. That is, the powerhead 50 provides the motive force to discharge fluid from each of the syringes 86a, 86b. One embodiment of what may be characterized as a syringe plunger drive assembly or syringe plunger driver is illustrated in FIG. 2C, is identified by reference numeral 56, and may be utilized by the powerhead 50 to discharge fluid from each of the syringes 86a, 86b. A separate syringe plunger drive assembly 56 may be incorporated into the powerhead 50 for each of the syringes 86a, 86b. In this regard and referring back to FIGS. 2A-B, the powerhead 50 may include hand-operated knobs 80a and 80b for use in separately controlling each of the syringe plunger drive assemblies 56.

Initially and in relation to the syringe plunger drive assembly 56 of FIG. 2C, each of its individual components may be of any appropriate size, shape, configuration and/or type. The syringe plunger drive assembly 56 includes a motor 58, which has an output shaft 60. A drive gear 62 is mounted on and rotates with the output shaft 60 of the motor 58. The drive gear 62 is engaged or is at least engageable with a driven gear 64. This driven gear 64 is mounted on and rotates with a drive screw or shaft 66. The axis about which the drive screw 66 rotates is identified by reference numeral 68. One or more bearings 72 appropriately support the drive screw 66.

A carriage or ram 74 is movably mounted on the drive screw 66. Generally, rotation of the drive screw 66 in one direction axially advances the ram 74 along the drive screw 66 (and thereby along axis 68) in the direction of the corresponding syringe 86a/b, while rotation of the drive screw 66 in the opposite direction axially advances the ram 74 along the drive screw 66 (and thereby along axis 68) away from the corresponding syringe 86a/b. In this regard, the perimeter of at least part of the drive screw 66 includes helical threads 70 that interface with at least part of the ram 74. The ram 74 is also movably mounted within an appropriate bushing 78 that does not allow the ram 74 to rotate during a rotation of the drive screw 66. Therefore, the rotation of the drive screw 66 provides for an axial movement of the ram 74 in a direction determined by the rotational direction of the drive screw 66.

The ram 74 includes a coupler 76 that that may be detachably coupled with a syringe plunger coupler 94 of the syringe plunger 90a/b of the corresponding syringe 86a/b. When the ram coupler 76 and syringe plunger coupler 94 are appropriately coupled, the syringe plunger 90a/b moves along with ram 74. FIG. 2C illustrates a configuration where the syringe 86a/b may be moved along its corresponding axis 100a/b without being coupled to the ram 74. When the syringe 86a/b is moved along its corresponding axis 100a/b such that the head 96 of its syringe plunger 90a/b is aligned with the ram coupler 76, but with the axes 68 still in the offset configuration of FIG. 2C, the syringe 86a/b may be translated within a plane that is orthogonal to the axis 68 along which the ram 74 moves. This establishes a coupled engagement between the ram coupler 76 and the syringe plunger coupler 96 in the above-noted manner.

The power injectors 10, 40 of FIGS. 1 and 2A-C each may be used for any appropriate application, including without limitation for medical imaging applications where fluid is injected into a subject (e.g., a patient) and/or any appropriate medical diagnostic and/or therapeutic application (e.g., injection of chemotherapy, pain management, etc.). Representative medical imaging applications for the power injectors 10, 40 include without limitation computed tomography or CT imaging, magnetic resonance imaging or MRI, single photon emission computed tomography or SPECT imaging, positron emission tomography or PET imaging, X-ray imaging, angiographic imaging, optical imaging, and ultrasound imaging. The power injectors 10, 40 each could be used alone or in combination with one or more other components. The power injectors 10, 40 each may be operatively interconnected with one or more components, for instance so that information may be conveyed between the power injector 10, 40 and one or more other components (e.g., scan delay information, injection start signal, injection rate).

Any number of syringes may be utilized by each of the power injectors 10, 40, including without limitation single-head configurations (for a single syringe) and dual-head configurations (for two syringes). In the case of a multiple syringe configuration, each power injector 10, 40 may discharge fluid from the various syringes in any appropriate manner and according to any timing sequence (e.g., sequential discharges from two or more syringes, simultaneous discharges from two or more syringes, or any combination thereof). Multiple syringes may discharge into a common conduit (e.g., for provision to a single injection site), or one syringe may discharge into one conduit (e.g., for provision to one injection site), while another syringe may discharge into a different conduit (e.g., for provision to a different injection site). Each such syringe utilized by each of the power injectors 10, 40 may include any appropriate fluid (e.g., a medical fluid), for instance contrast media, therapeutic fluid, a radiopharmaceutical, saline, and any combination thereof. Each such syringe utilized by each of the power injectors 10, 40 may be installed in any appropriate manner (e.g., rear-loading configurations may be utilized; front-loading configurations may be utilized; side-loading configurations may be utilized).

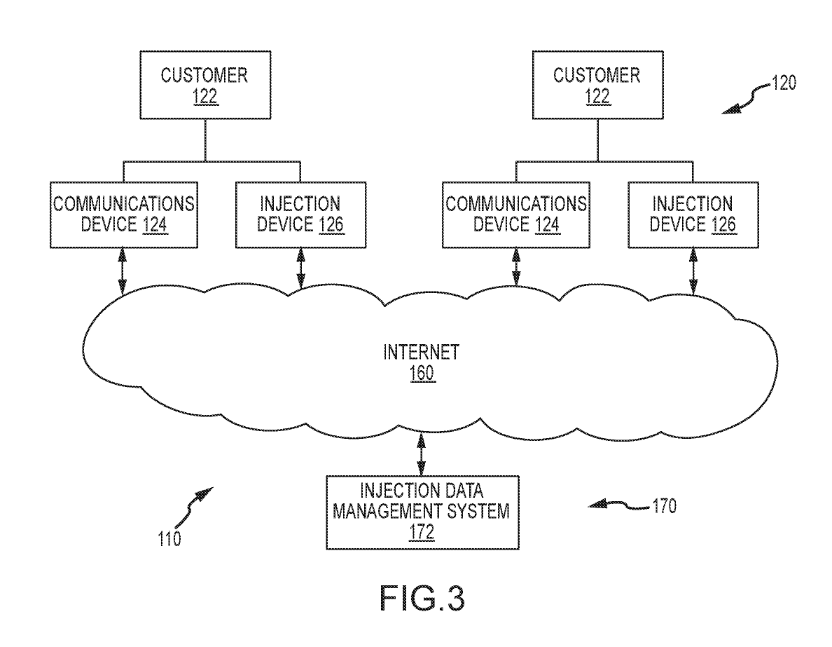

One embodiment of injection data management architecture is illustrated in FIG. 3 and is identified by reference numeral 110. The injection data management architecture 110 utilizes an owner/operator side 170 and a customer side 120. The owner/operator side 170 includes what may be characterized as an injection data management system 172. Generally, the injection data management system 172 stores injection-related data on a customer-by-customer basis, and is accessible over the Internet 160 on a customer-by-customer basis (e.g., via one or more Internet addresses for the injection data management system 172). Multiple customers 122 may access the injection data management system 172 through a common Internet address. In any case, the injection data management system 172 may be characterized as a centralized repository for injection-related data, and customers 122 may be viewed as remotely accessing injection-related data being stored on the injection data management system 172 over the Internet 160.

The customer side 120 of the architecture 110 may include one or more customers 122. Any appropriate number of customers 122 may be on the customer side 120 of the architecture 110. Customers 122 may be characterized as owners or operators of facilities such as clinics, hospitals, stand-alone imaging centers, mobile imaging vehicles (e.g., vans, trucks, tractor-trailers), or the like. At least one injection device 126 is utilized by each customer 122. Any appropriate number of injection devices 126 may be utilized by each customer 122. The injection devices 126 for a given customer 122 may be disposed at one or more locations of the operations of the customer 122. Each injection device 126 for each customer 122 may be of any appropriate type (e.g., a power injector). A given customer 122 could utilize a multiple types of injection devices 126. All injection devices 126 could also be the same for one or more customers 122. A given customer 122 could have a single injection device 126.

Injection-related data relating to the operation of each injection device 126 of each customer 122 may be transmitted to the injection data management system 172 over the Internet 160 (e.g., by associating injection-related data that is output to the Internet 160 with an Internet address for the injection data management system 172). This may be characterized as “uploading” customer injection-related data to the injection data management system 172 (e.g., a transmission of customer injection-related data in an external-to-internal direction—from outside the injection data management system 172 to the injection data management system 172). The injection-related data relating to the operation of a given injection device 126 could be transmitted directly from the injection device 126 in any appropriate manner and on any appropriate basis. Alternatively, injection-related data relating to the operation of a given injection device 126 could initially be transmitted to a data storage device of any appropriate type, and then transmitted from this data storage device to the injection data management system 172 over the Internet 160. A separate or external data storage device could be operatively interconnected with any appropriate number of injection devices 126.

Injection-related data that is generated in relation to the operation of an injection device 126 may be of any appropriate type. For instance, such injection-related data may be in the form of a programmed and achieved fluid injection rate, a date of an injection, a programmed and achieved volume of fluid (e.g. contrast media) injected, a type of fluid injected, contrast media usage, minimum/average/maximum fluid pressure generated during an injection, fluid injection rate as a function of time during an injection, injection pressure as a function of time during an injection, a lot number of the fluid injected, an iodine-content of the fluid injected, an identity of the facility responsible for manufacturing and/or packaging the fluid injected, a manufacturing date of the fluid injected, an expiration date of the fluid injected, the National Drug Code (NDC) of the fluid injected, a volume of fluid remaining and subsequently discarded after an injection procedure (e.g., contrast media remaining in a syringe at the completion of an injection procedure), or any combination thereof. With regard to the minimum, average, and maximum pressures noted above, the same may relate to the pressure that was generated during a single or multiple injection procedures (e.g., executed in accordance with a first injection protocol or a programmed protocol) using a particular injection device 126.

At least some of this injection-related data may be retrieved directly from the associated injection device 126, at least some of this injection-related data may be retrieved other than from the injection device 126, or any combination thereof. For instance and in the latter regard, a data reader of any appropriate type (e.g., an RFID reader) may be used to read one or more data tags (or other data storage devices) associated with one or more fluid containers (e.g., syringes) that may be used by the injection device 126. In one embodiment, such a data reader may be used to identify one or more aspects of the fluid (e.g., type, concentration, preparation date, initial volume of fluid in the fluid container) to be delivered by the injection device 126. The injection data management system 172 may also be configured to allow customers 122 to store other injection-related data, for instance quantities of contrast media that have been ordered and/or received by a customer 122.

Injection-related data from a given customer 122 may be transmitted to the injection data management system 172 over the Internet 160 on any appropriate basis. For instance, injection-related data associated with operation of a given injection device 126 could be transmitted to the injection data management system 172 over the Internet 160 immediately or shortly after completion of an injection protocol or procedure using this device 126. Another option would be to delay transmission of injection-related data associated with operation of a given injection device 126 to the injection data management system 172 for some period of time. For instance, “batches” of injection-related data associated with operation of one or more injection devices 126 from a given customer 122 could be transmitted to the injection data management system 172 over the Internet 160 on any appropriate basis (e.g., daily; periodically; ad hoc; in accordance with a schedule).

Each customer 122 in the architecture 110 has at least one communications device 124 for communicating with the injection data management system 172 over the Internet 116. In this regard, the injection data management system 172 may have one or more Internet addresses, and at least one Internet address is available for each of the communications devices 124 of a given customer 122 for communicating with the injection data management system 172. A given customer 122 may utilize one or more types of communications devices 124. Representative communications devices 124 include without limitation laptop computers, netbooks, desktop computers, tablet PCs, personal digital assistants, smart phones, or the like. Generally, each customer 122 may gain access to the injector data management system 172 via an Internet address (e.g., each communications device 124 for each customer 122 may be characterized as being outside of or external to the injector data management system 172), and again multiple customers 122 could in fact each access the injector data management system 172 through a common Internet address.

Customers 122 may view their injection-related data that has been previously stored on the injection data management system 172 using any appropriate communications devices 124 for the Internet 160 (e.g., by addressing a request for injection-related data to the injection data management system 172). Customers 122 may submit a request for an injection-related data selection to the injection data management system 172 using a communications device 124 (e.g., such a request may include one or more Internet addresses to which the request is targeted). Such a request for a customer injection-related data selection may be characterized as allowing a customer 122 to access, review, analyze, and/or compile all or any portion of its own injection-related data. All results of any such request may be characterized as a “customer injection-related data selection.”

Representative customer injection-related data selections include without limitation: 1) the number of injection procedures run on one or more of the injection devices 126 of a given customer 122, at one or more of the facilities 140 of a given customer 122, or both; 2) the number of a particular type of injection procedure (e.g., a particular injection protocol) run on one or more of the injection devices 126 of a given customer 122, at one or more of the facilities 140 of a given customer 122, or both; 3) the flow rate (minimum, maximum, and/or average) used for one or more (e.g., a particular type of) injection procedures run on one or more of the injection devices 126 of a given customer 122, at one or more of the facilities 140 of a given customer 122, or both; 4) the average fluid injection rate employed by one or more of the injection devices 126 of a given customer 122, or optionally specific for various injection procedures; 5) the actual and/or average volumes of fluid (e.g. contrast media) utilized by one or more of the injection devices 126 of a given customer 122 for a given time period (e.g., daily, weekly, monthly, quarterly, yearly); 6) the volume of fluid remaining and subsequently discarded after an injection procedure (e.g., contrast media remaining in a syringe at the completion of an injection procedure); 7) the pressure (minimum, maximum, and/or average) generated during the execution of a particular type of injection procedure (e.g., in accordance with a first injection protocol or otherwise) run on a particular one of the injection devices 126 of a given customer 122; and 8) the pressure (minimum, maximum, and/or average) generated during the execution of multiple injection procedures (e.g., each executed in accordance with a common injection protocol or otherwise) run on one or more of the injection devices 126 of a given customer 122, at one or more of the facilities 140 of a given customer 122, or both.

A request for a customer injection-related data selection that is output to the Internet 160, in association with an Internet address for the injection data management system 172 (e.g., directed to one or more Internet addresses), may be of any appropriate type and/or form. The request may be in the form of one or more filter selections, parameters, or the like for purposes of defining the desired customer injection-related data selection. An electronic form (e.g., viewable on a communications device 124 and using one or more web-based applications and/or a web-based interface from the injection data management system 172) may be used by a customer 122 to formulate a request for the desired customer injection-related data selection. A customer 122 could request injection-related data on one or more injection devices 126 at one or more of its facilities. A customer 122 could also request injection-related data on one or more of its facilities (e.g., contrast media usage at one or more of its facilities; contrast media inventory at one or more of its facilities).

A customer injection-related data selection that is accessed from the injector data management system 172 by a customer 122 may be viewed as being downloaded from the injection data management system 172 (e.g., a transmission of customer injection-related data in an internal-to-external direction, or from inside the injection data management system 172 to outside the injection data management system 172). A customer injection-related data selection that is received by a customer 122 in response to a request may be of any appropriate type and in any appropriate form (e.g., an electronic report viewable on a communications device 124 and using one or more web-based applications and/or a web-based interface of the injection data management system 172). Any such customer injection-related data selection may be of any appropriate content as well. For instance, a customer injection-related data selection may be customer contrast media usage data (e.g., how much contrast media has been used by one or more injection devices), may be customer contrast media inventory data (e.g., how much contrast media remains in inventory at one or more facilities owned and/or operated by a given customer), or both. Any such customer injection-related data selection could be specific to a single injection device 126 or could be based upon one or more injection devices 126 at the same location/facility, one or more injection devices 126 at different locations/facilities, or both. That is, a given customer injection-related data selection may not only be based upon first injection-related data relating to operation of a first injection device 126, but second injection-related data relating to operation of a second injection device 126 as well.

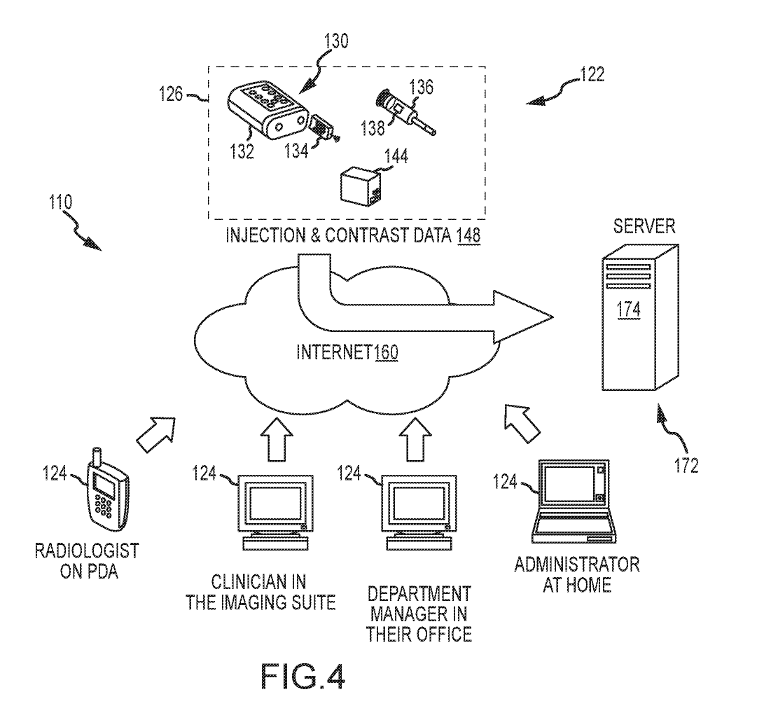

A representative implementation of the injection data management architecture 110 for a given customer 122 is presented in FIG. 4. The illustrated injection device 126 is in the form of a power injector 130 having a powerhead 132. The powerhead 132 of the power injector 130 accommodates two syringes 136 (only one shown in FIG. 4). Each syringe 136 may include a data storage tag 138. Any appropriate information may be stored on this data storage tag 138, and may be retrieved in any appropriate manner using a data reader 134. In one embodiment, the data storage tag 138 is in the form of an RFID tag 138, and the data reader 134 is in the form of an RFID reader 134. The data reader 134 could be incorporated in any appropriate manner by the power injector 130, or could be a completely separate unit from the power injector 130. Injection and contrast data 148 relating to operation of the injection device 126 may be transmitted to a server 174 of the injection data management system 172 over the Internet 160 (e.g., the injection and contrast data 148 may be sent to one or more Internet addresses).

FIG. 4 also presents a number of representative communications devices 124 that may be utilized by a customer 122 to communicate with the injection data management system 172, including without limitation: 1) a radiologist using a PDA 124; 2) a clinician (e.g., in an imaging suite) using a desktop computer 124; 3) a department manager (e.g., in their office) using a desktop computer 124; and 4) administrator (e.g., in their home) using a laptop computer 124. Any appropriate personnel of a customer 122 may communicate with the injection data management system 172 over the Internet 160 using any appropriate data communications device 124 and from any appropriate location.

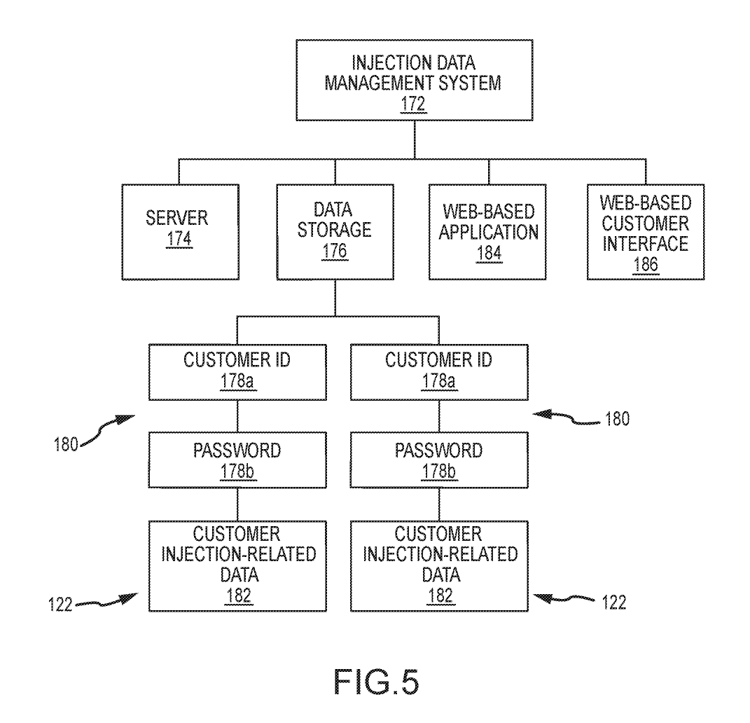

The injection data management system 172 may be of any appropriate configuration to store injection-related data for multiple customers 122, and to allow each customer 122 to retrieve injection-related data in any appropriate manner over the Internet 160. A representative configuration for the injection data management system 172 is presented in FIG. 5. The injection data management system 172 is illustrated as including one or more servers 174 of any appropriate type (e.g., associated with one or more Internet addresses) and distributed in any appropriate manner, one or more data storage devices 176 of any appropriate type and distributed in any appropriate manner, one or more web-based applications 184, and a web-based customer interface 186.

Two customers 122 are illustrated in FIG. 5. As noted above, any appropriate number of customers 122 may be associated with the injection data management system 172. Each customer 122 may have a customer ID or username 178a that may be stored on or in relation to the injection data management system 172 (e.g., in association with the injection-related data 182 of the customer 122). The customer ID 178a may be of any appropriate type and in any appropriate form for purposes of identifying a customer 122 to the injection data management system 172 (e.g., alphanumeric). Each customer 122 may also have an associated password 178b of any appropriate type/form (e.g., alphanumeric) that is stored on or in relation to the injection data management 172. The customer ID 178a and password 178b for a customer 122 may be collectively characterized as authentication data 180, and which may be stored on the injection data management system 172 in association with the injection-related data 182 of the associated customer 122.

It should be appreciated that the authentication data 180 may be of any appropriate type and/or form (e.g., both a customer ID and password 178b may not be required in all instances—a single “input” could define the authentication data 180). In the illustrated example, a customer 122 may have to enter two pieces of information to gain access (over the Internet 160) to its customer injection-related data 182 that is stored on the injection data management system 172, for instance both a customer ID 178a and password 178b. In any case, if the authenticating data provided by customer 122 (over the Internet 160) matches the corresponding authentication data 180 stored on the injection data management system 172, the customer 122 may then gain access to its own customer injection-related data 182 on the system 172 over the Internet 160. Preferably, each customer 122 is only able to access its own injection-related data 182 stored on the injection data management system 172 over the Internet 160. For instance, a first customer 122 should not be able to access the injection-related data 182 of a second customer 122 on the injection data management system 172 over the Internet 160.

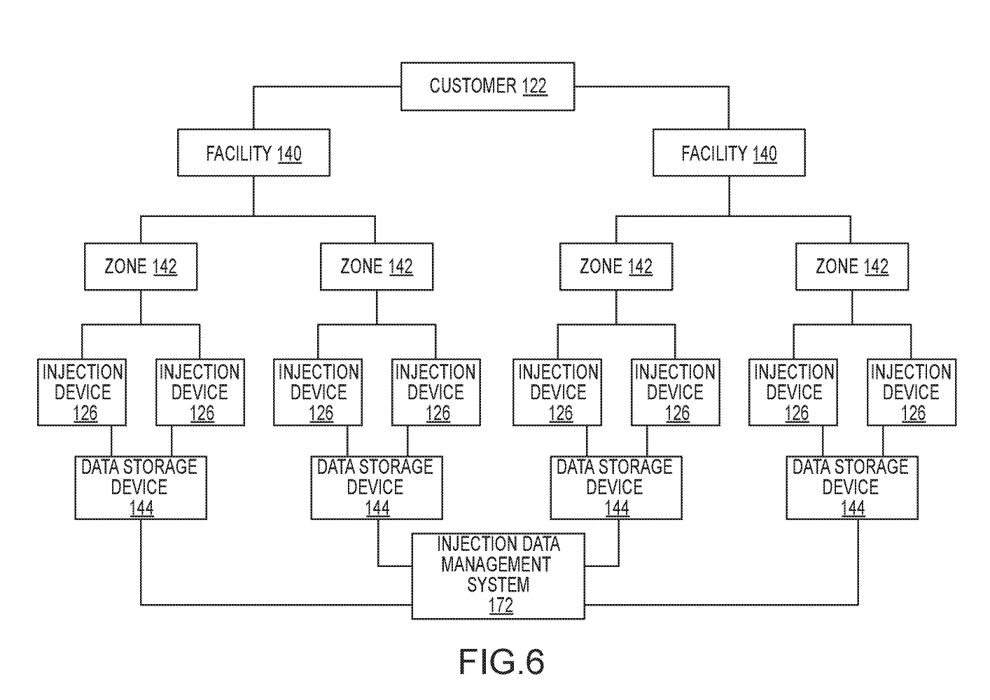

FIG. 6 is a schematic in accordance with the injection data management architecture 110 of FIG. 3, and illustrates the case of a given customer 122 having multiple facilities 140. Here, the customer 122 is illustrated as having two facilities 140. A given customer 122 may of course have any appropriate number of facilities 140. Each customer facility 140 includes one or more zones 142 (two in the illustrated embodiment). Each zone 142 may be defined in any appropriate manner. For instance, a first zone 142 may be a first imaging suite (or a first wing of a clinic), while a second zone 142 may be a second imaging suite (or a second wing of a clinic). In any case, each zone 142 may utilize one or more injection devices 126. In the illustrated embodiment, each injection device 126 in a given zone 142 provides its injection-related data to a common data storage device 144. Each data storage device 144 in each customer zone 142 may be utilized by any appropriate number of injection devices 126. Each data storage device 144 is operatively interconnected with the injection data management system 172 via the Internet 160 for purposes of transmitting injection-related data to the injection data management system 172 in the above-noted manner (e.g., to one or more Internet addresses).

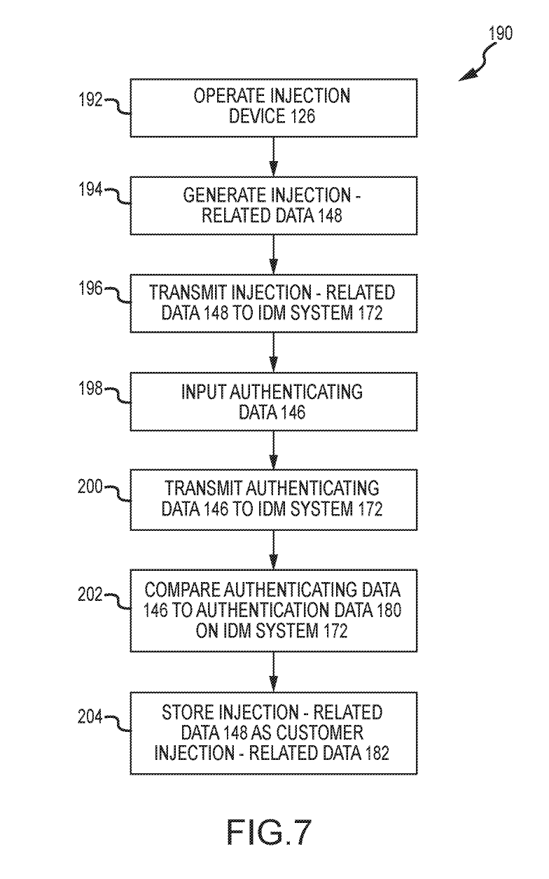

FIG. 7 illustrates one embodiment of what may be characterized as a data storage protocol 190—a way of transmitting injection-related data from a customer 122 to the injection data management system 172 over the Internet 160. The protocol 190 includes operating an injection device 126 (step 192). Injection-related data 148 is generated in relation to this operation of the injection device 126 (step 194). Injection-related data 148 is transmitted over the Internet 160 to the injection data management system 172 (e.g., the data 148 is directed to one or more Internet addresses) pursuant to step 196 of the data storage protocol 190. The customer 122 may also input authenticating data 146 (e.g., a customer ID/username 178a and/or password 178b) using a communications device 124 pursuant to step 198. The authenticating data 146 is also transmitted to the injection data management system 172 (step 200) over the Internet 160 (e.g., the authenticating data 146 is directed to one or more Internet addresses). The authenticating data 146 (input by the customer 122) is compared with the authentication data 180 (stored on the injection data management system 172) by the injection data management system 172 pursuant to step 202 of the protocol 190. If the authenticating data 146 (step 198) matches the authentication data 180 (step 202), the injection-related data 148 from step 194 may be stored on the injection data management system 172 as customer injection-related data 182 (step 204). That is, the injection-related data 148 associated with step 194 may be stored on the injection data management system 172 in association with a specific customer 122. It should be appreciated that steps 196-200 may be executed in any appropriate order.

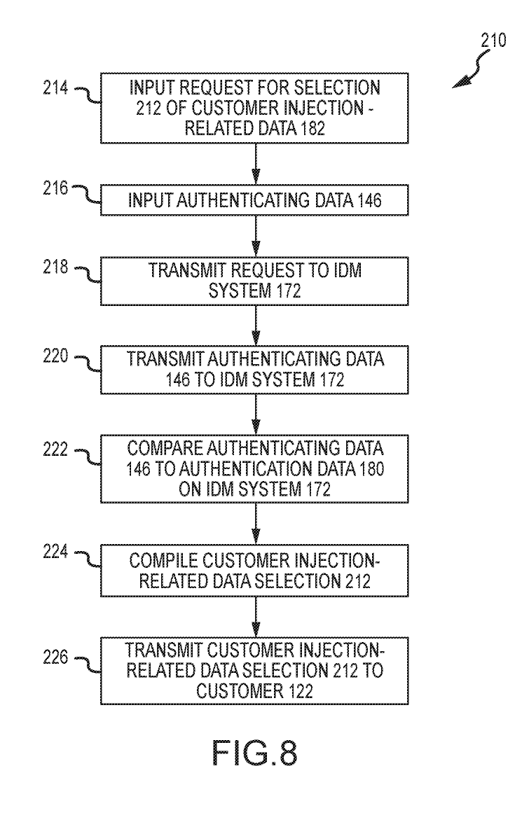

One embodiment of what may be characterized as an injection-related data request protocol is illustrated in FIG. 8 and is identified by reference numeral 210—a way for a customer 122 to view and/or assess its injection-related data on the injection data management system 172 over the Internet 160. The protocol 210 includes inputting a request for a selection 212 of customer injection-related data 182 (step 214). Authenticating data 146 (e.g., customer ID 178a and/or password 178b) may be input by the customer 122 pursuant to step 216. Steps 214 and 216 may be executed by a customer 122 using one or more communications devices 124. Both the request for a selection 212 of customer injection-related data 182 (step 214) and the authenticating data 146 (step 216) are transmitted to the injection data management system 172 (via steps 218 and 220, respectively) over the Internet 160. Step 222 is directed to the injection data management system 172 comparing the authenticating data 146 (step 216) with the authentication data 180 stored on the injection data management system 172. If the authenticating data 146 (step 216) matches authentication data 180 stored on the injection data management system 172, the injection data management system 172 compiles a customer injection-related data selection 212 pursuant to step 224. This customer injection-related data selection 212 may then be transmitted to the customer 122 (step 226) over the Internet 160.

Representative customer injection-related data selections 212 for purposes of step 214 of the injection-related data request protocol 210 of FIG. 8 include without limitation: 1) the number of injection procedures run on one or more of the injection devices 126 of a given customer 122, at one or more of the facilities 140 of a given customer 122, or both; 2) the number of a particular type injection procedure (e.g., a particular injection protocol) run on one or more of the injection devices 126 of a given customer 122, at one or more of the facilities 140 of a given customer 122, or both; 3) the flow rate (minimum, maximum, and/or average) used for one or more (e.g., a particular type of) injection procedures run on one or more of the injection devices 126 of a given customer 122, at one or more of the facilities of a given customer 122, or both; 4) the average fluid injection rate employed by one or more of the injection devices 126 of a given customer 122, or optionally specific for various injection procedures; 5) the actual and/or average volumes of fluid (e.g. contrast media) utilized by one or more of the injection devices 126 of a given customer 122 for a given time period (e.g., daily, weekly, monthly, quarterly, yearly); 6) the volume of fluid remaining and subsequently discarded after an injection procedure (e.g., contrast media remaining in a syringe at the completion of an injection procedure); 7) the pressure (minimum, maximum, and/or average) generated during the execution of a particular type of injection procedure (e.g., in accordance with a first injection protocol or otherwise) run on a particular one of the injection devices 126 of a given customer 122; and 8) the pressure (minimum, maximum, and/or average) generated during the execution of multiple injection procedures (e.g., each executed in accordance with a common injection protocol or otherwise) run on one or more of the injection devices of a given customer 122, at one or more of the facilities 140 of a given customer 122, or both.

The request for a customer injection-related data selection 212 (step 214 of the protocol 210 of FIG. 8) that is transmitted to the injection data management system 172 over the Internet 160 may be of any appropriate type and/or form. This request may be formulated/submitted using one or more communications devices 124 on the customer side 120, and using the web-based customer interface 186 and one or more web-based applications 184 on the owner/operator side 170. The request may be in the form of one or more filter selections, parameters, or the like for purposes of defining the desired customer injection-related data selection 212. An electronic form (e.g., via one or more web-based applications 184 and/or the web-based interface 186 from the injection data management system 172) may be used by a customer 122 on a communications device 124 to formulate a request for the desired customer injection-related data selection 212. A customer 122 could request injection-related data on one or more injection devices 126 at one or more of its facilities 140. A customer 122 could also request injection-related data on one or more of its facilities (e.g., contrast media usage at one or more of its facilities 140; contrast media inventory at one or more of its facilities 140).

The injection data management system 172 will compile the customer injection-related data selection 212 (step 224) in accordance with the above-noted request (step 214), and will then transmit the selection 212 to the customer 122 over the Internet 160. A customer injection-related data selection 212 that is received by a customer 122 in response to such a request (step 214) may be of any appropriate type and in any appropriate form (e.g., an electronic report viewable through one or more web-based applications 184 and/or the web-based interface 186, using a communications device 124). Any such customer injection-related data selection 212 may be of any appropriate content as well. For instance, a customer injection-related data selection 212 may be customer contrast media usage data (e.g., how much contrast media has been used by one or more injection devices 126), may be customer contrast media inventory data (e.g., how much contrast media remains in inventory at one or more facilities 140 owned and/or operated by a given customer 122), or both. Any such customer injection-related data selection 212 could be specific to a single injection device 126 or could be based upon one or more injection devices 126 at the same location/facility 140, one or more injection devices 126 at different locations/facilities 140, or both. That is, a given customer injection-related data selection 212 may not only be based upon injection-related data relating to operation of a first injection device 126, but second injection-related data relating to operation of a second injection device 126 as well.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

What is claimed is:

1. A method, comprising:
operating a first injection device that is located at a first facility operated by a first customer;
generating first injection data that is associated with said operating a first injection device step;
outputting said first injection data and addressed to an injection data system;
outputting a first request for a first customer injection data selection and addressed to said injection data system, wherein said first customer injection data selection is specific to said first customer;
entering authenticating data; and
receiving said first customer injection data selection in response to said first request, wherein said receiving step is only executed when said authenticating data matches first authentication data that is associated only with first customer injection data on said injection data system, wherein said first customer injection data is both specific to said first customer and comprises said first injection data, wherein said first customer injection data selection is based on at least a portion of said first customer injection data, and wherein said first authentication data being specific to said first customer injection data precludes injection data from all other customers on said injection data system from being included in said first customer injection data selection, said injection data from said all other customers on said injection data system being associated with other injection devices located at other facilities that are operated by said all other customers.

2. An injection data system, comprising:
one or more data storage devices;
first customer injection data stored on said one or more data storage devices and in association with a first customer that operates a first facility comprising a first injection device;
first customer authentication data stored on said one or more data storage devices and associated with said first customer injection data, wherein access to said first customer injection data on said one or more data storage devices requires receipt of customer authenticating data by said injection data system that matches said first customer authentication data;
second customer injection data stored on said one or more data storage devices and in association with a second customer that operates a different second facility comprising a second injection device;
second customer authentication data stored on said one or more data storage devices and associated with said second customer injection data, wherein access to said second customer injection data on said one or more data storage devices requires receipt of customer authenticating data by said injection data system that matches said second customer authentication data; and
a web-based customer interface associated with said one or more data storage devices, wherein receipt of customer authenticating data by said injection data system that matches said first customer authentication data only provides access to said first customer injection data that is stored on said one or more data storage devices and does not provide access to injection data of any other customers on said one or more data storage devices, and wherein receipt of customer authenticating data by said injection data system that matches said second customer authentication data only provides access to said second customer injection data that is stored on said one or more data storage devices and does not provide access to injection data of any other customers on said one or more data storage devices.

3. The system of claim 2, further comprising at least one server in communication with said one or more data storage devices.

4. The system of claim 2, further comprising at least one web-based application in communication with said one or more data storage devices.

5. The system of claim 2, wherein said first and second customer injection data each relate to an injection procedure and are each selected from a programmed fluid injection rate, an achieved fluid injection rate, date of injection, time of injection, a programmed volume of fluid to be injected, an achieved volume of said injected fluid, a type of fluid injected, contrast media usage, customer contrast media inventory data, a minimum fluid pressure that was generated, a maximum fluid pressure that was generated, an average fluid pressure that was generated, fluid injection rate as a function of time, injection pressure as a function of time, a lot number of said injected fluid, an iodine content of said injected fluid, an identity of a facility responsible for manufacturing said injected fluid, an identity of a facility responsible for packaging said injected fluid, a manufacturing date of said injected fluid, an expiration date of said injected fluid, the National Drug Code associated with said injected fluid, a volume of fluid remaining for use by said injection device upon completion of said injection procedure, and any combination thereof.

6. The system of claim 2, wherein said first customer authentication data comprises at least one of first user name and a first password, and wherein said second customer authentication data comprises at least one of second user name and a second password.

7. A method, comprising:
receiving first injection data at an injection data system, wherein said first injection data relates to a first injection device disposed a first facility that is operated by a first customer;
storing said first injection data on said injection data system;
associating first customer authentication data with all first customer injection data that is stored on said injection data system, wherein said first customer injection data is both specific to said first customer and comprises said first injection data;
receiving second injection data at said injection data system, wherein said second injection data relates to a second injection device disposed at a second facility that is operated by a second customer;
storing said second injection data on said injection data system, wherein said first and second customers are different from one another;
associating second customer authentication data with all second customer injection data that is stored on said injection data system, wherein said second customer injection data is both specific to said second customer and comprises said second injection data; and
providing access to customer injection data stored by said injection data system on a customer-by-customer basis such that receipt by said injection data system of first customer authenticating data that matches said first customer authentication data only provides access to said first customer injection data that is stored by said injection data system and does not provide access to injection data of any other customers on said injection data system, and such that receipt by said injection data system of second customer authenticating data that matches said second customer authentication data only provides access to said second customer injection data that is stored by said injection data system and does not provide access to injection data of any other customers on said injection data system.

8. The method of claim 7, wherein said first injection data and second injection data each relate to an injection procedure in which a fluid is injected and are each selected from a programmed fluid injection rate, an achieved fluid injection rate, date of injection, time of injection, a programmed volume of fluid to be injected, an achieved volume of said injected fluid, a type of said injected fluid, contrast media usage, a minimum fluid pressure that was generated, a maximum fluid pressure that was generated, an average fluid pressure that was generated, fluid injection rate as a function of time, injection pressure as a function of time, a lot number of said injected fluid, an iodine content of said injected fluid, an identity of a facility responsible for manufacturing said injected fluid, an identity of a facility responsible for packaging said injected fluid, a manufacturing date of said injected fluid, an expiration date of said injected fluid, the National Drug Code associated with said injected fluid, a volume of fluid remaining for use by said injection device upon completion of said injection procedure, and any combination thereof.

9. The method of claim 7, wherein said injection data system comprises at least one server on the Internet.

10. The method of claim 7, wherein said receiving first injection data step and said receiving second injection data step each comprise utilizing the Internet.

11. The method of claim 7, wherein said storing said first injection data step comprises linking said first injection data with said first customer on said injection data system, and wherein said storing second injection data step comprises linking said second injection data with said second customer on said injection data system.

12. The method of claim 7, wherein said providing access step comprises restricting access to customer injection data, that is stored by said injection data system in association with said first customer, to said first customer, and wherein said providing access step further comprises restricting access to customer injection data, that is stored by said injection data system in association with said second customer, to said second customer.

13. The method of claim 7, further comprising:
providing at least one web-based application for communicating with said injection data system.

14. The method of claim 7, further comprising:
providing a web-based interface for communicating with said injection data system.

15. The method of claim 7, further comprising:
granting said first customer access to said injection data system such that access to an entirety of said second customer injection data is prohibited; and
granting said second customer access to said injection data system such that access to an entirety of said first customer injection data is prohibited.

16. The method of claim 7, further comprising:
receiving a first request for a first customer injection data selection over the Internet and at said injection data system;
outputting said first customer injection data selection via the Internet and in response to said first request, wherein an entirety of said first customer injection data selection is based on at least a portion of said first customer injection data.

17. The method of claim 16, further comprising:
receiving a second request for a second customer injection data selection over the Internet and at said injection data system;
outputting said second customer injection data selection via the Internet and in response to said second request, wherein an entirety of said second customer injection data selection is based on at least a portion of said second customer injection data.

18. The method of claim 17, wherein said second customer injection data selection comprises data that relates to at least one injection procedure performed by said second customer.

19. The method of claim 17, wherein said second customer injection data selection is specific to one injection device.

20. The method of claim 17, wherein said second customer injection data selection pertains to a plurality of different injection devices.

21. The method of claim 16, wherein said first customer injection data selection comprises data that relates to at least one injection procedure performed by said first customer.

22. The method of claim 16, wherein said first customer injection data selection is specific to one injection device.

23. The method of claim 16, wherein said first customer injection data selection pertains to a plurality of different injection devices.
